(19) United States
(12) Patent Application Publication
(10) Pub. No.: US 2025/0339265 A1
(43) Pub. Date: Nov. 6, 2025

(54) SYSTEMS, DEVICES AND METHODS FOR REPLACEMENT VALVES COMPRISING UNIBODY STENT STRUCTURES

(71) Applicant: Capstan Medical Inc., Santa Cruz, CA (US)

(72) Inventors: Spencer C. Noe, Santa Cruz, CA (US); Daniel T. Wallace, Santa Cruz, CA (US); Ian Mahaffey, Santa Cruz, CA (US); Mitasha Malhan, Santa Cruz, CA (US)

(21) Appl. No.: 18/871,750

(22) PCT Filed: Jun. 8, 2023
(86) PCT No.: PCT/US2023/068136

§ 371 Date: Dec. 4, 2024

Related U.S. Application Data
(60) Provisional application No. 63/350,207, filed on Jun. 8, 2022.

(51) Int. Cl. A61F 2/24

(52) U.S. Cl. CPC A61F 2/2418; A61F 2220/0016

(57) ABSTRACT

The embodiments herein are directed to replacement valve comprising a double-wall, folded stent structure with an inner wall providing an inner lumen and a valve structure that is attached to a stent structure. The inner wall is spaced apart from an outer wall that is configured to seal and/or anchor to the surround native valve anatomy, but is contiguous with the inner wall via a transition wall. The transition wall may result from the folding, inversion or eversion of a single tubular structure into a double-wall unibody tubular stent structure. The stent structure is configured to reversibly collapse into a collapsed configuration exhibiting reduced diameter or reduced cross-sectional shape for loading into a catheter and for delivery to a target anatomical site and an expanded configuration.

### CROSS-REFERENCE TO RELATED APPLICATIONS

This application claims benefit under 35 U.S.C. § 119(e) to U.S. Provisional Application Ser. No. 63/350,207, filed Jun. 8, 2022, which is hereby incorporated by reference in its entirety.

### BACKGROUND

This patent application relates generally to the treatment of valvular diseases, and more specifically to methods and apparatus for minimally invasive tricuspid valve replacement.

Valvular heart disease is a significant burden to patients and healthcare systems, with a prevalence of 2-3% worldwide, and with an increasing prevalence in aging populations. Valvular disease typically results from cardiovascular causes such as myocardial infarction and heart failure, but may also result from a variety of etiologies, comprising autoimmune, infective and degenerative causes. The etiology of valvular disease also varies with the affected valve. For example, tricuspid valve regurgitation may be caused by congenital disease, infective endocarditis or rheumatic fever, iatrogenic events such as injuries from pacemaker wires or endomyocardial biopsy, Marfan syndrome, and other issues.

### BRIEF SUMMARY

Further growth of transcatheter tricuspid valve therapies is challenged by the difficulty by tricuspid valve anatomy and physiology, compared to more established transcatheter aortic and mitral valve therapies. For example, the anatomy of and around the tricuspid valve is less firm than the anatomy of and around the aortic and mitral valves which makes securing replacement valves to the tricuspid valve difficult.

To address these issues, embodiments described herein are directed to a replacement heart valve comprising a unibody, folded, double-wall stent, with a stent cover and a valve structure (e.g., leaflet valve) attached to the inner lumen of the stent. The double wall stent structure decouples or reduces the effect on the geometry of the retention structure on the geometry of the valve support. This comprises external forces acting through the valve annulus during the cardiac cycle, as well as the effect of non-circular valve annulus shapes. The double-wall stent structure also allows the valve support to have a different size and shape from outer annulus support, without the valve support having to expand or deform against the native anatomy, or to at least partially isolate effects from expansion of the outer annulus support against the anatomy. The unibody design may also permit a greater structural integrity by reducing complications relating to force concentrations between joined. welded or mechanically connected support components and/or their attachment in situ.

In an embodiment, a replacement heart valve is disclosed. The replacement heart valve comprises a unibody stent structure. The stent structure comprises a collapsed configuration and an expanded configuration. The stent structure also comprises an outer wall comprising an enlarged diameter region and a reduced diameter region. Additionally, the stent structure comprises an inner wall defining an inner lumen, and a transition wall between the outer wall and the inner wall. The replacement heart valve also comprises a valve structure located in the inner lumen of the inner wall. The unibody stent structure further comprises a plurality of longitudinal struts and a plurality of lateral struts integrally formed together, each longitudinal strut contiguously located along the inner wall, transition wall and a portion of the outer wall. The outer wall may have a generally flared or frustoconical shape, with the later diameter located at one end opposite of the transition wall. The inner wall may have a generally cylindrical shape. For embodiments comprising a replacement tricuspid valve, the longitudinal struts may be provided in multiples of three, e.g, a total of three, six, nine or twelve longitudinal struts. In some variants. the longitudinal struts extend along the entire length of the inner wall, and the length of the transition wall and the entire length of the outer wall. In other variants, however, the longitudinal struts only extend partially along the length of the outer wall. The length of the longitudinal strut segment in the outer wall may be shorter, the same as, or longer than the length of the longitudinal strut segment in the inner wall. While the inner wall and transition wall may comprise a longitudinally non-foreshortening configuration, the outer wall may be partially longitudinally foreshortening and non-foreshortening, with the non-foreshortening portion being contiguous with the transition wall, and the foreshortening portion located at the free end of the outer wall. Radially extending anchor struts may also be provided. The anchor struts may be curved radially outward and may be located in the foreshortening portion of the outer wall.

In one embodiment, a replacement heart valve is provided, comprising a unibody stent structure that comprises a collapsed configuration and an expanded configuration, an outer wall comprising an enlarged diameter region and a reduced diameter region, an inner wall defining an inner lumen, a transition wall between the outer wall and the inner wall, and a valve structure located in the inner lumen of the inner wall, wherein the unibody stent structure further comprises a plurality of longitudinal struts and a plurality of lateral struts integrally formed together, each longitudinal strut contiguously located along the inner wall, transition wall and a portion of the outer wall, wherein a ratio of an axial length of a portion of the outer wall without any of the plurality of longitudinal struts and an axial length of the portion of the outer wall with at least some of the plurality of longitudinal struts is in the range of 1:1 to 1:1.5. The valve may be a tricuspid replacement valve. The transition wall may be downstream of the enlarged diameter region. The outer wall may comprise a first region extending from the transition wall and a second region extending from an open end of the outer wall, the first region comprising the plurality of longitudinal struts and the second region free of the plurality of longitudinal struts. The first region may comprise at least one of the plurality of lateral struts and the second region may comprise at least one of the plurality of lateral struts, the at least one of the plurality of lateral struts of the first region exhibiting a strut configuration that is different than the at least one of the plurality of lateral struts of the second region. The at least one of the plurality of lateral struts of the first region comprise legs that are generally linear with deformations near the end of each leg, and wherein the at least one of the plurality of lateral struts of the second region comprise legs exhibiting a generally S-like shape or combined concave/convex shape. At least a portion of the first region of the outer wall may be configured to be disposed in a ventricle of a heart and at least a portion of the second region of the outer wall may be configured to be disposed in an atrium of the heart. The second region of the outer wall may be configured to be more flexible than the first region of the outer wall. The outer wall may comprise a plurality of barbs extending therefrom. The outer wall may comprise a first region extending from the transition wall and a second region extending from an open end of the outer wall, the first region comprising the plurality of longitudinal struts and the second region free of the plurality of longitudinal struts, and wherein the plurality of barbs extend from the second region of the outer wall. The plurality of barbs may be oriented more towards an outer opening of the outer wall than towards the transition wall. The plurality of longitudinal struts and the plurality of lateral struts may comprise nitinol. The replacement heart valve may further comprise a skirt material disposed on at least a portion of the outer wall, at least a portion of the inner wall, and at least a portion of the transition wall. The skirt material may comprises a first material and a second material that is different than the first material. The first material may comprise a weave material and the second material may comprise a knit material. The weave material may be disposed at least a portion of the inner wall and at least a portion of the outer wall extending from an outer opening of the outer wall, a portion of the weave material extending between the inner wall and the outer wall, and wherein the knit material is disposed on at least a portion of the transition wall and a portion of the outer wall extending from the transition wall. The portion of the weave material extending between the inner wall and the outer wall may extend across the outer opening. The portion of the weave material extending between the inner wall and the outer wall ay extend across an intermediate location that is spaced from the outer opening. The outer wall may comprise a plurality of barbs extending therefrom, and the skirt material may comprise a plurality of openings formed therein, each of the plurality of openings configured to receive one of the plurality of barbs. The skirt material may comprise one or more lead openings therein configured to allow one or more electrical leads to pass therethrough. The ratio of the axial length of the portion of the outer wall without any of the plurality of longitudinal struts and the axial length of the portion of the outer wall with at least some of the plurality of longitudinal struts may be in the range of 1:1.0 to 1:1.4. An inflow angle between an inlet of the outer wall and an inlet of the inner wall may be in the range of 5 degrees to 35 degrees, or in the range of 25 degrees to 35 degrees. A ratio between a diameter of the inner wall and a diameter of the outer wall at an endpoint of at least one of the plurality of longitudinal struts may be in the range of 1:1 to 1:2. The replacement heart valve of claim 24, wherein the ratio between the diameter of the inner wall and the diameter of the outer wall at the endpoint of at least one of the plurality of longitudinal struts may be in the range of 1.4 to 1.6. The transition wall may have an average radius of curvature in the range of about 1 mm to 5 mm, or about 1.5 mm to 3 mm. The ratio of an axial dimension of a combined inner wall and transition wall to the axial dimension of a combined outer wall and transition wall may be in the range of about 1:1 to 1:1.5, or 1.1 to 1.3. A ratio of an axial dimension of the inner wall to an axial dimension of the outer wall may be in a range of about 1:05 to 1:1.4, or about 1.1 to 1.3. A ratio between a diameter of the outer wall comprising at least one end of the plurality of longitudinal struts and a maximum diameter of the outer wall may be in a range of 1:1 to 1:1.5, or 1:1.2 to 1:1.4.

### BRIEF DESCRIPTION OF THE DRAWINGS

FIG. 1A is a partial schematic side elevation of one embodiment of a stent structure with the rear half of the stent structure omitted: FIG. 1B is a schematic top plan view of the stent structure: FIG. 1C is a partial cross-sectional view of the inner stent structure of FIG. 1A without the outer wall: FIG. ID is a partial schematic side elevation view of the outer wall of the stent structure, without the inner wall: and FIG. 1E is a schematic component view of two longitudinal struts from FIG. 1A: FIGS. IF to II are isolated cross-sectional side profile views of the stent structure in FIG. 1A depicting various exemplary dimensions of the stent structure.

FIGS. 2A and 2B depict various exemplary strut configurations:

FIG. 3 is a schematic cross-sectional views of a replacement valve with a skirt material attached to a stent structure, according to an embodiment:

FIG. 4 is a schematic cross-sectional views of a replacement valve with a skirt material attached to a stent structure, according to an embodiment:

FIG. 5 is a schematic isometric view of a replacement valve with a skirt material defining one or more lead openings, according to an embodiment:

FIG. 6 is a schematic isometric view of a replacement valve with a valve structure, according to an embodiment: side elevation view of another embodiment of a heart valve stent with the leaflet valve and skirt attached:

FIGS. 7A and 7B are schematic cross-sectional views of an exemplary views of a deployment procedure for a heart replacement valve and delivery system.

### DETAILED DESCRIPTION

The embodiments herein are directed to replacement valve comprising a double-wall, folded stent structure with an inner wall providing an inner lumen and a valve structure that is attached to a stent structure. The inner wall is spaced apart from an outer wall that is configured to seal and/or anchor to the surround native valve anatomy, but is contiguous with the inner wall via a transition wall. The transition wall may result from the folding, inversion or eversion of a single tubular structure into a double-wall unibody tubular stent structure. The stent structure is configured to reversibly collapse into a collapsed configuration exhibiting reduced diameter or reduced cross-sectional shape for loading into a catheter and for delivery to a target anatomical site and an expanded configuration.

In further embodiments, the outer wall of the stent structure may be shaped with an enlarged diameter region and a reduced diameter region downstream from the enlarged diameter region. The enlarged diameter region and the reduced diameter region may facilitate anchoring of the stent structure across the desired anatomical site. The reduced diameter region is configured to expand against the native valve leaflets and/or anatomical orifice, while the enlarged diameter region provides mechanical interference or resistance to displacement. The mechanical and/or friction interference may anchor the stent structure to the anatomy and form a seal that prevents flow of fluid between the stent structure and the anatomy. In an embodiment, the outer wall does not comprise an additional enlarged diameter region downstream from the reduced diameter region since the additional enlarged diameter region may interfere with the cords of the tricuspid valve or other anatomy.

Although some of the exemplary embodiments described herein are directed to transcatheter replacement of tricuspid valves, the components and structures herein are not limited to any specific valve or delivery method, and may be adapted to implantation at the tricuspid, pulmonary, aortic valve locations, and also in non-cardiac locations (e.g., the aorta, venous system or cerebrospinal fluid system, or a native or artificial conduit, duct or shunt). As used herein, the spatial references to a first or lower end of a component may also be characterized by the anatomical space the component occupies and/or the relative direction of fluid flow. For example, the first or lower end of stent structure of a replacement tricuspid valve may also be referenced as the ventricular end or downstream end of the valve, while the opposite end (e.g., second or upper end) may be referenced as the atrial end or upstream end of the valve.

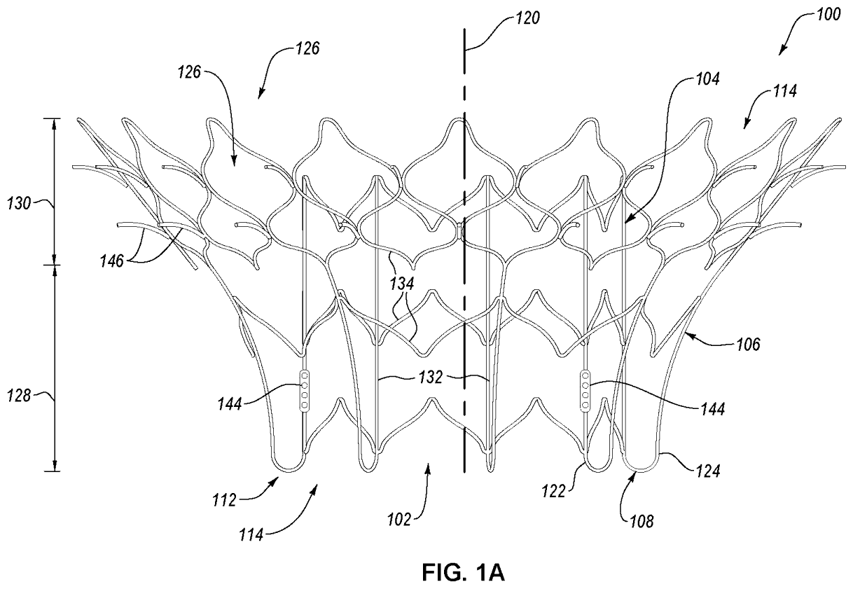

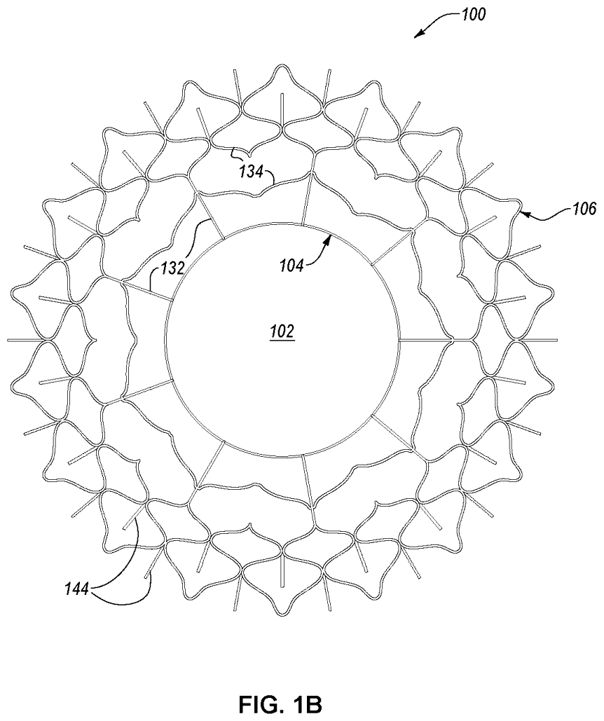

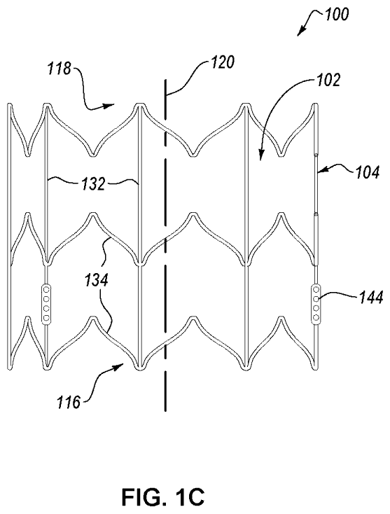

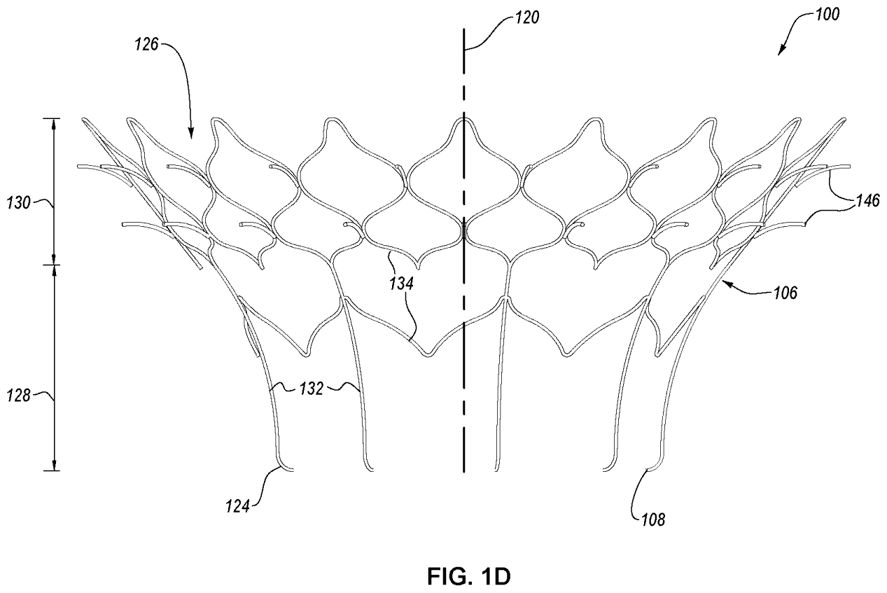

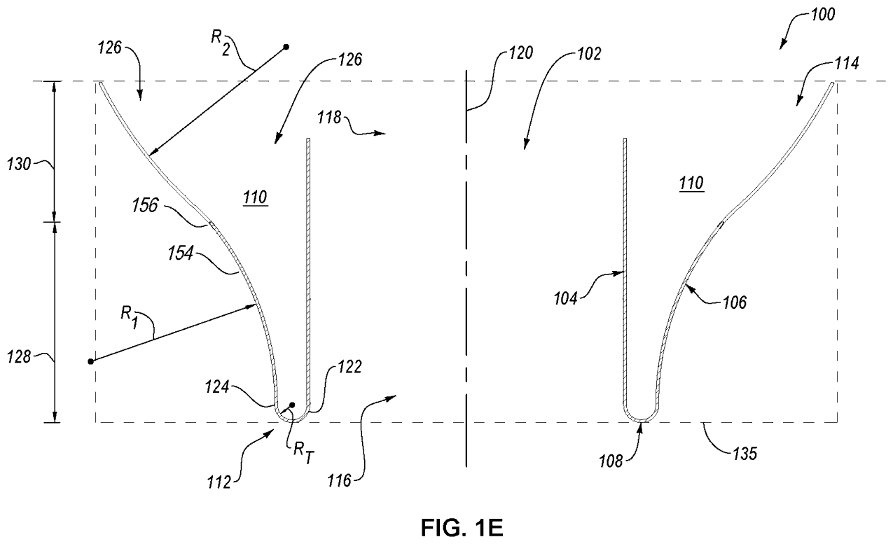

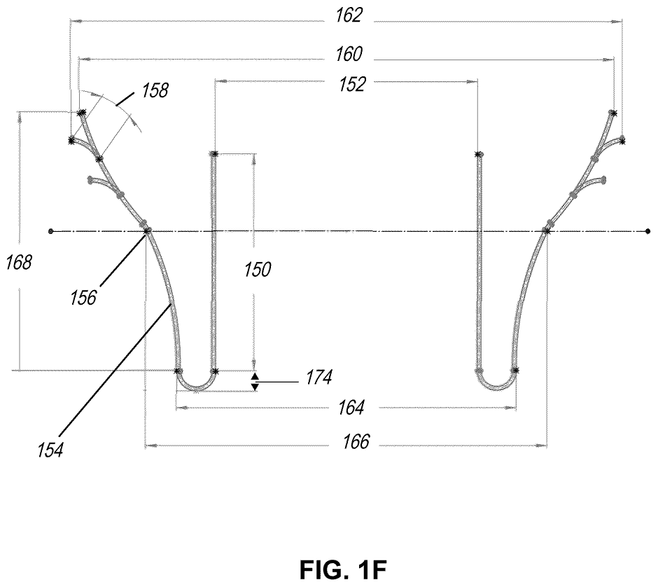

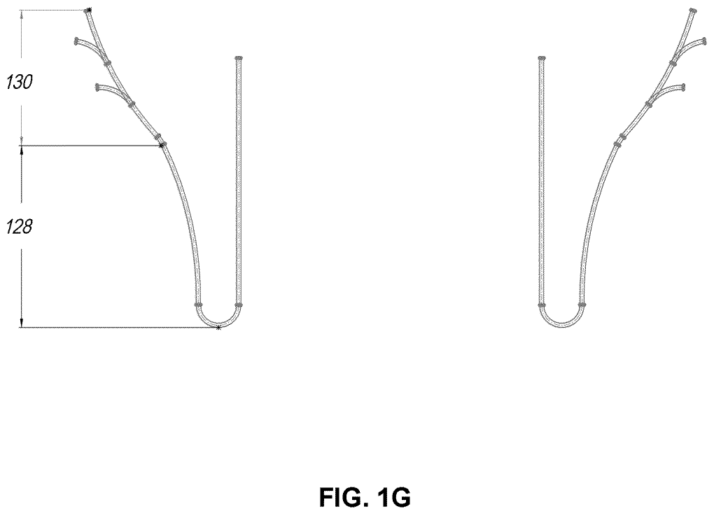

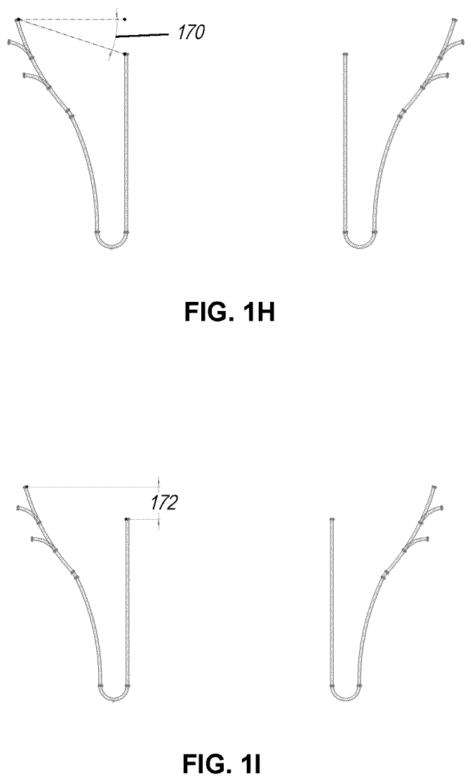

An exemplary embodiment of a stent structure 100 is depicted in FIGS. 1A-1E with the stent structure 100 in its expanded configuration. For illustrative reasons, the back half of the stent structure 100 illustrated in FIGS. 1A. IC, and ID have been omitted to simplify the depiction of the stent structure. The stent structure 100 comprises an inner lumen 102 formed by an inner wall 104. An outer wall 106 is spaced radially apart from the inner wall 104 via a transition wall 108. and forms an annular cavity 110. The stent structure 100 has first closed end 112 that is located at the transition wall 108, and a second open end 114 of the outer wall 106, wherein the annular cavity 110 is open and accessible. The stent structure 100 may exhibit a unibody structure (e.g., formed from a single piece) which provides a structural integrity to the stent structure 100 that better redistributes forces acting on the stent structure 100, with less force concentration found typically found in stent structures that comprises multiple components.

The inner lumen 102 comprises a first opening 116 surrounded by the transition wall 108 and a second opening 118 at the second open end 114 of the stent structure 100. The longitudinal axis 120 of the inner lumen 102 is typically coincident with the central axis of the stent structure 100, but in some variations, the inner lumen 102 may be eccentrically located relative to the outer wall 106 of the stent structure 100. The inner lumen 102 typically comprises a circular cross-sectional shape with a generally cylindrical shape between the first opening 116 and second opening 118, as depicted in FIGS. 1A-1C. In other examples, the inner lumen 102 may comprise a frustoconical, oval or polygonal shape. In some variations, the stent structure 100 may comprises an inner lumen where the size and/or shape of the first and second openings 116, 118 may be different. Referring to FIG. 1F, the length 150 of the inner lumen 102 may be measured from the first opening 116 to the second opening 118, and may be in the range of 10 mm to 50 mm, 15 mm to 40 mm, 20 mm to 25 mm, 15 mm to 20 mm, 17.5 mm to 22.5 mm, 20 mm to 25 mm, 22.5 mm to 27.5 mm, 25 mm to 30 mm, 27.5 mm to 32.5 mm, 30 mm to 35 mm, 32.5 mm to 27.5 mm, or 30) about 35 mm to 40 mm, or 22 to 27 mm, and the diameter 152 or maximum cross-sectional dimension of the inner lumen 102 may be in the range of 15 mm to 40 mm, 15 mm to 25 mm, 20 mm to 30 mm, 25 mm to 35 mm, or 27 mm to 32 mm. In embodiments where the inner lumen 102 comprises a non-cylindrical shape, the difference between the diameter or cross-sectional dimension of the first opening 116 and the second opening 118 may be in the range of 1 mm to 10 mm, 1 mm to 5 mm, or 1 mm to 3 mm.

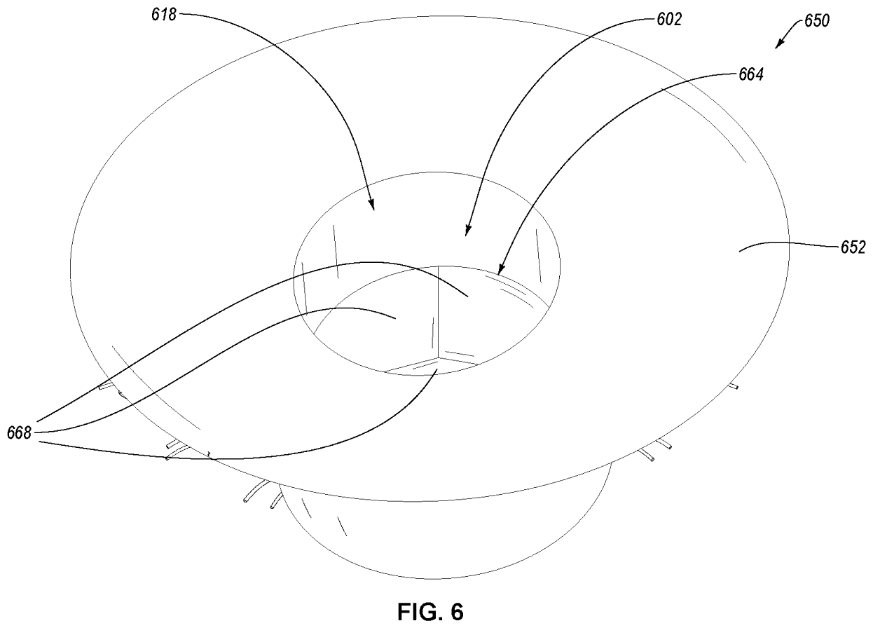

The maximum length L1 may be selected based on the size of the anatomy and is selected to be sufficiently large to allow the valve structure (discussed in more detail with regards to FIG. 6) to function within the inner lumen 102. However, in some variations, it may be generally desired to minimize the maximum length 150 to limit the length of the stent structure 100 when the stent structure 100 is in the collapsed configuration to make it easier to insert the stent structure 100 into the desired anatomy. Also, it may be desirable to minimize the maximum length 150 to decrease the length of the stent structure 100 that is disposed in the ventricle since the portions of the stent structure 100 that are disposed in the ventricle may interfere with ventricle.

The location of the first and second openings 116, 118 of the inner lumen 102 relative to the overall stent structure 100 may also vary. In some variations, the first opening 116 of the inner lumen 102 may be recessed relative to the first end 112, as depicted in FIGS. 1A and 1E. In other examples, the first opening 116 may be generally flush with the first end 112 of the transition wall 108 of the stent structure 100. The location of the first opening 116 may also be characterized as recessed, flush or protruding relative to the longitudinal location of the inner junction 122 between the inner wall 104 or lumen 102 and transition wall 108, or relative to the outer junction 124 between the transition wall 108 and the outer wall 106. Likewise, the second opening 118 of the inner lumen 102 may also be characterized as recessed, flush or protruding, relative to the longitudinal location of outer opening 126 of the outer wall 106. For example, the second opening 118 of the inner lumen 102 comprises an offset or protruding location relative to the outer opening 126 of the outer wall 106. In some variations, the inner lumen 102 may protrude relative to the outer opening 126 of the outer wall 106 in variations where a smaller or shorter outer wall 106 is preferred to accommodate smaller size native valve anatomy. The size of the inner lumen 102, however, may remain relatively the same size between different size variations, to provide consistent valve geometry and/or hemodynamic characteristics.

The transition wall 108 of the stent structures 100 has a generally annular and rounded shape (e.g., concave or convex shape) surrounding the inner lumen 102 in the expanded configuration. but in other variations may have a different shape and/or surface angle. For example, the transition wall 108 on cross section may comprise a rounded (e.g., semi-circular) shape between the inner junction 122 and the outer junction 124, but in other variations, may comprise a generally linear shape (e.g., exhibiting a generally orthogonal angle relative to the longitudinal axis 120 of the inner lumen 102). Referring to FIG. 1E, the transition wall 108 of stent structure 100 may exhibit an average radius of curvature R_{T}. The average radius of curvature R_{T} may be in the range of 0.5 mm to 1.5 mm, 1 mm to 2 mm. 1.5 mm to 2.5 mm, 1.5 mm to 2 mm, 1.5 mm to 3 mm, 2 mm to 3 mm 2.5 mm to 3.5 mm, 1 mm to 5 mm, or 3 mm to 4 mm.

Referring to FIG. IF, the maximum diameter 160 of the outer wall 106 in its maximally expanded configuration without the barbs 146, which is also the diameter of the inlet or outer opening of the outer wall 106, may be in the range of 40 mm to 80 mm, 45 mm to 70 mm, 50 mm to 70 mm, 55 mm to 65 mm, or 58 mm to 62 mm. Including the distal tips of the barbs 146, the maximum diameter 162 of the outer wall 106 in its maximally expanded configuration may be in the range of 40 mm to 80 mm, 50 mm to 75 mm, 55 mm to 65 mm, 60 mm to 65 mm, or 60 mm to 70 mm. The minimal diameter 164 of the outer wall 106 in its maximally expanded configuration, which may also be the diameter at the junction of the outer wall 106 and the transition wall 108, may be in the range of 25 mm to 60 mm, 30 mm to 50 mm, 30 mm to 45 mm, or 35 to 40 mm. Referring back to FIG. 1E, the diameter 166 of the outer wall 106 at the junction between the first and second regions 128. 130, which may also be the inflection point of the concave/convex shape of the outer wall 106, may be in the range of 25 mm to 60 mm, 30 mm to 55 mm, 35 mm to 50 mm, 40 mm to 50 mm, 45 to 50 mm, 40 mm to 45 mm, or 42 to 47 mm. The axial length 168 of the outer wall 106 may be in the range of 25 mm to 30 mm, 27 mm to 32 mm, 24 mm to 35 mm, or 26 mm to 34 mm. The axial length 174 of the transition wall 108 may be in the range of 2 mm to 3 mm, 2.0 mm to 2.5 mm, 1 mm to 5 mm, 2 mm, to 4 mm, or 2 mm to 8 mm. In other variations, however, the outer wall 106 may comprise a generally straight wall configuration on cross-section, i.e. a cylindrical or frusto-conical shape.

Referring to FIG. 1G, the axial dimension of the first region 128 of the outer wall 106 and the axial dimension of the second region 130 of the outer wall 106 may vary, depending on the desired relative implantation level of the valve 100 relative to the annulus. The axial dimension of the first region 128 of the outer wall 106, as measured parallel to the longitudinal axis of the valve 100, may be in the range of 6 mm to 20 mm, 8 mm to 18 mm, 10 mm to 15 mm, or 12 mm to 15 mm. The axial dimension of the second region 130 of the outer wall 106, as measured parallel to the longitudinal axis of the valve 100, may be in the range of 16 mm to 20 mm, 15 mm to 20 mm, 12 mm to 24 mm, or 10 mm to 28 mm. A ratio of the axial length of the second portion, or the portion of the outer wall without any of the plurality of longitudinal struts, and the first portion, or an axial length of the portion of the outer wall with at least some of the plurality of longitudinal struts is in the range of 1:1 to 1:1.5, 1:1.2 to 1:1.4, or 1:1.3 to 1:1.4. The ratio of an axial dimension of a combined inner wall 104 and transition wall 108 to the axial dimension of a combined outer wall 106 and transition wall 108 may be in the range of about 1:1 to 1:1.5, 1:05 to 1:1.4, 1:1.1 to 1:1.2, or 1:1.15 to 1.20, or 1:1.2 to 1:1.3. The relative difference in axial dimension of the outer wall 106 and the inner wall may be in the range of −5 mm to +15 mm, −2 mm to +12 mm, 0 mm to +8 mm, +1 mm to +5 mm, or +2 mm to +4 mm, for example. The ratio of wall lengths of the inner wall 104 to the outer wall 106 (excluding the transition wall 108) may be in the range of 1:0.8 to 1:2, 1:1 to 1:1.8, 1:1 to 1:1.5, 1:1.1 to 1:1.4, 1:1.1 to 1:1.3 1:1.2 to 1:1.4, for example. The ratio between a diameter 166 of the outer wall 106 at the junction of the first and second portions of the outer wall 106, and the maximum diameter 160 of the outer wall 106 may be in a range 1:1 between 1:1.5. 1:1.2 to 1:1.4, 1:1.3 to 1:1.4, or 1:1.2 to 1:1.3.

Referring to FIG. 1H, the inlet or inflow angle formed by the opening 126 of the outer wall 106 and the inlet opening 118 of the inner wall 104, or the longitudinal axis 120 of the stent 100, may be in the range of 15 degrees to 20 degrees, 16 degrees to 20 degrees, 14 degrees to 22 degrees, 16 degrees to 19 degrees, 5 degrees to 35 degrees. 10 degrees to 25 degrees, 12 degrees to 25 degrees, 20 degrees to 30 degrees. 25 degrees to 35 degrees or 25 degrees to 30 degrees. In the embodiment depicted in FIG. 1E, where the cross-sectional configuration of the outer wall 106 is non-linear, the inflow angle of the outer wall 106 may be defined by the second region 130 of the outer wall 106, e.g, from the longitudinal midpoint or the inflection point of the outer wall 106 to the lip or opening of the outer wall 106. In some variations, it may be beneficial for the second region 130 of the outer wall 106 to comprise a concave configuration relative to the inner wall 104 so that the immediate region of the outer wall 106 about the opening 126 is oriented relatively closer to the longitudinal axis 120 than to a transverse orientation to the longitudinal axis 120. Referring to FIG. 1I, the axial length differential 172 between the opening 126 of the outer wall 106 and the inlet opening 118 of the inner wall 104 may be between 4 mm and 6 mm, 4 mm and 5 mm, 4 mm and 8 mm, or 3 mm and 6 mm. Referring back to FIG. 1F, the ratio between the diameter 152 of the inner wall 104 to the diameter 166 of the outer wall 106 at the junction of the first and second portions 128, 130 of the outer wall 106 (or at the terminal end of a longitudinal strut 154, may be in the range of 1:1 to 1:2, 1:1.4 to 1:1.6, 1:1.5 to 1:1.6, 1:1.3 to 1:1.7, or 1:1.2 to 1:1.8. The ratio between the diameter 152 of the inner wall 104 to the maximum diameter 160 of the outer wall may be in the range of 1:1.5 to 1:3, 1:1.7 to 1.2.7. 1:1.8 to 1:2.5, 1:1.9 to 1:2.2, or 1.9 to 1:2.1, for example.

As noted previously, in some embodiments, the outer wall 106 of the stent structure 100 comprises a non-cylindrical shape when in the expanded configuration. This may include a flared or frustoconical shape. The outer wall 106 may comprise a first region 128 that is contiguous with the transition wall 108 and a second region 130 that forms the outer opening 126. The first region 128 may exhibit a concave curvature and the second region 130 may exhibit a convex curvature relative to an exterior of the stent structure 100 (e.g., a location not within the inner lumen 102 or the annular cavity 110) adjacent to the outer wall 106. The first region 128 may comprise the reduced diameter region of the stent structure 100 thereby allowing at least a portion of the first region 128 to expand against the native valve leaflets and/or anatomical orifice. The reduced diameter region of the stent structure 100 may be at or extend from a portion of the outer wall 106 at or near the outer junction 124 which may prevent or at least inhibit the stent structure 100 from interfering with anatomy downstream from the outer junction 124. The second region 130 may comprise the enlarged diameter region of the stent structure 100 thereby allowing at least a portion of the second region 130 to provide mechanical interference or resistance to displacement. The enlarged diameter region of the stent structure 100 may be at or extend from a portion of the outer wall 106 at or near the outer opening 126. In an example, the second region 130 may be used to anchor the stent structure 100 in the atrium above a tricuspid valve and to form a seal in the atrium which prevents or at least inhibits back flow of blood from the ventricle to the atrium. In one example, the boundary between the first region 128 and the second region 130 may be the nominal or expected location of an annulus of a valve, such as the annulus of the tricuspid valve.

In an embodiment, as shown in FIG. 1E, the first region 128 may exhibit a first average radius of curvature R₁ and the second region 130 may exhibit a second average radius of curvature R₂. The average radiuses of curvature R₁ and R₂ may be independently selected to be 20 mm to 30 mm, 25 mm to 35 mm, 30 mm to 40 mm, 35 mm to 45 mm, 40 mm to 50 mm, 45 mm to 55 mm, 50 mm to 60 mm, 55 mm to 65 mm, or 60 mm to 70 mm. In an embodiment, at least one of the first region 128 or the second region 130 may be substantially linear.

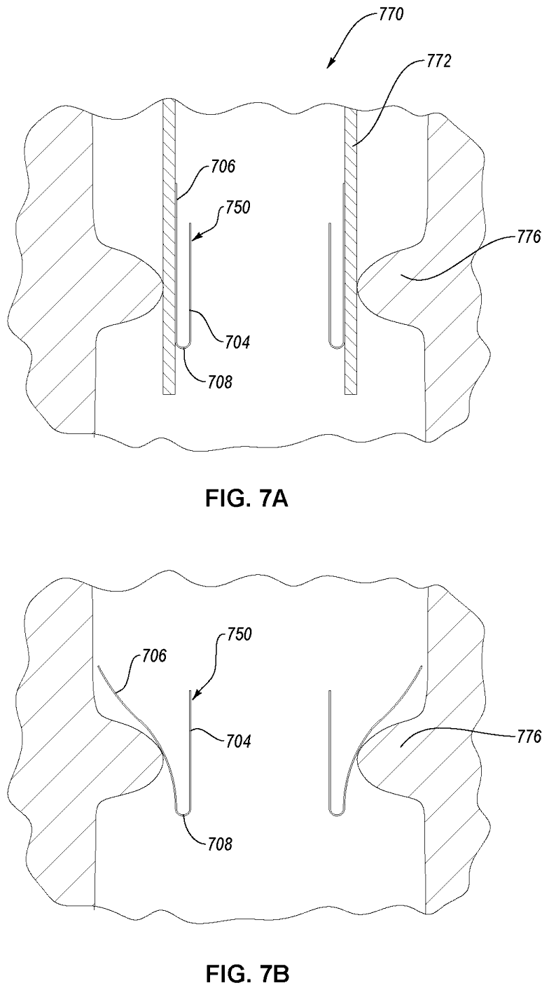

The average radii of curvatures of the stent structure 100 may be used to define the geometry of the stent in the expanded configuration, but also affect the geometry of the stent in its delivery or collapsed configuration (shown in FIG. 7A). Regions or segments of the stent may be configured with a smaller average radius of curvature to facilitate the folding of the stent at that region or segment as the stent is collapsed for the collapsed configuration. Regions of segments of the stent may be configured with a larger average radius of curvature to facilitate straightening of that region or segment for the collapsed configuration. For example, with stent structure 100, a relatively smaller radius of curvature R_{T} facilitates the folding or collapsing of the stent structure around the transition wall 108, while a larger radii of curvatures R₁ and R₂ facilitates the flattening of the first region 128 and the second region 130, respectively, during delivery or loading of the device into the delivery system.

The non-cylindrical configuration of the outer wall 106 may allow the outer wall 106 to exhibit more foreshortening than the inner wall 104 as the outer wall 106 transitions from a relatively straight orientation in the collapsed configuration to the concave/convex orientation in its expanded configuration. In some variations, the longitudinal shift upon expansion of the portions of the first region 128 at or adjacent to (e.g., within 10 mm, within 5 mm, or within 3 mm) the reduced diameter region of the outer wall 106 may be less than 5 mm, 4 mm, 3 mm, 2 mm, or 1 mm. In some variations, the longitudinal shift upon expansion of the portions of the second region 130 at or adjacent to (e.g., within 10 mm, within 5 mm, or within 3 mm) the enlarged diameter region of the outer wall 106 may be greater than 5 mm, 10 mm, 15 mm, or 20 mm.

It is noted that the general funnel-like shape (e.g., non-hourglass-like shape) of the outer wall 106 formed by the first and second regions 128, 130 may facilitate attachment of the stent to anatomy that does not lie in a plane, like the tricuspid valve. For example, outer walls exhibiting an hourglass shape may be used to anchor a stent to anatomy that is in a plane, such as a mitral valve. However, anchoring a stent exhibiting the hourglass shape to non-planar anatomy may cause tilting of the stent which, in turn, may cause the stent to interfere with adjacent anatomy. Meanwhile, it is believed that the funnel-like shape of the outer wall 106 does not exhibit such issues.

The stent structures herein disclosed further comprise a plurality of integrally formed stent struts segments, as depicted in FIGS. 1A to 1D. Some struts may be characterized as longitudinal strut segments 132 or lateral strut segments 134. The longitudinal strut segments 132 generally reside within a radial plane 135 (shown schematically as a dashed box in FIG. 1E) in which the longitudinal axis 120 also resides, where the two longitudinal strut segments 132 are lying in different adjacent radially oriented planes. Lateral strut segments 134 are integrally formed with the longitudinal strut segments 132. Lateral strut segments 134 generally reside within a tangential plane relative to the radial plane 135 (e.g., the lateral strut segments 134 extend generally within a curved surface of a cylinder or funnel). In embodiments with an even number of equally spaced apart longitudinal struts, as depicted in FIG. 1E, each radial plane 135 will comprise the longitudinal axis 120 of the stent structure 100, and two longitudinal strut segments 132 located on opposite sides of the stent structure 100.

Contiguous longitudinal strut segments 132 form a longitudinal strut 154. Each longitudinal strut extends along at least a portion of at least one wall (e.g., at least one of the inner wall 104, the outer wall 106, or the transition wall 108). In an embodiment, each longitudinal strut extends along an entirety of the inner wall 104 (e.g., from the first opening 116 to the inner junction 122), an entirety of the transition wall 108 (e.g., from the inner junction 122 to the outer junction 124), and along a portion or an entirety of the outer wall 106. In such an embodiment, the longitudinal strut provide structural integrity and better redistributes stress to the inner wall 104, the transition wall 108, and the portion of the outer wall 106 that comprise the longitudinal strut. In some variations, the first region 128 of the outer wall 106 may also comprise a portion and a terminal end 156 of the longitudinal struts 154 in the outer wall 106, while the second region 130 of the outer wall 106 may lack any of the longitudinal struts. Meanwhile, the portion of the outer wall 106 that does not comprise the longitudinal strut may exhibit greater flexibility than the portion of the outer wall 106 that comprises the longitudinal strut. The portions of the outer wall with and without the longitudinal struts may also be characterized as comprising a longitudinally non-foreshortening portion being contiguous with the transition wall, and a foreshortening portion located at the free end of the outer wall. The greater flexibility of the portion of the outer wall 106 that does not comprise the longitudinal strut may facilitate greater expansion of such portions of the outer wall 106 when switching the stent structure 100 from the collapsed configuration to the expanded configuration. In certain embodiments, the length of the longitudinal strut segment in the outer wall may be shorter, the same as, or longer than the length of the longitudinal strut segment in the inner wall. In other variants, the length of the longitudinal strut segment in the outer wall may be characterized as a percentage of the overall longitudinal length of the outer wall, e.g. from 25% to 100%, 30% to 75%, 40% to 60%, and the like.

In a particular example, the first region 128 of the outer wall 106 comprises the longitudinal struts while the second region 130 of the outer wall 106 does not comprise the longitudinal struts. The location of the outer wall 106 where the longitudinal struts terminate may be the expected location of an annulus of a valve (e.g., the annulus of the tricuspid valve) when the stent structure 100 is disposed in the valve. In such an example, the diameter of the second region 130 is able to increase more than the diameter of the first region 128 when the stent is expanded at the implantation site. The greater flexibility of the portion of the outer wall 106 that does not comprise the longitudinal struts may allow the portion of the outer wall 106 that does not comprise the longitudinal struts to better conform to the adjacent anatomy than the portions of the outer wall 106 that comprise the longitudinal struts. In an embodiment, at least one longitudinal strut may be provided along the entire folded length of a stent structure 100 (e.g., along the length of the inner wall 104, through the transition wall 108, and along the length of the outer wall 106). Further, the foreshortening of the first region 128 and the second region 130 depends, at least in part, on the presence of the longitudinal struts 132. For example, the longitudinal struts cause the first region 128 to exhibit little to no foreshortening when switching from the expanded configuration to the collapsed configuration. The limited foreshortening of the first region 128 prevents or at least minimizes the length increase of the stent structure 100 (measured parallel to the longitudinal axis 120) when switching the stent structure 100 from the expanded configuration to the collapsed configuration which, in turn, makes it easier to insert the stent structure 100 into anatomy without interfering with the anatomy. The lack of the longitudinal struts segments 132 in the second region 130 causes the second region 130 to exhibit foreshortening when switching the stent structure 100 from the expanded configuration to the collapsed configuration. It is noted that the foreshortening of the second region 130 is less likely to adversely interfere with anatomy (e.g., of the tricuspid valve) than the first region 128 since the second region 130 may be used to interact with the anatomy to anchor the stent structure 100.

In exemplary stent structure 100, the longitudinal strut segments 132 along the inner lumen 102 of the stent structure 100 comprise a linear configuration, so the longitudinal strut segments 132 are generally parallel in both their expanded and contracted configurations. Because of this arrangement, the inner lumen 102 may not exhibit or may exhibit limited foreshortening when changing from the contracted to the expanded configuration. This may reduce or eliminate any axial stretching of the valve structure attached to the inner lumen 102. This may also permits the inner lumen 102 to be predictably positioned and deployed while reducing the risk of inadvertent position shifting.

The stent structure 100 may comprise any suitable number of longitudinal struts. In an example, the number of longitudinal struts is a multiple of the number of leaflets that form the valve. In such an example, the number of longitudinal struts allows each leaflet to be equally supported thereby preventing or at least inhibiting unequal wear between the leaflets which may cause the valve structure to fail. For instance, the valve comprising the stent structure 100 may comprise three leaflets when the valve is a tricuspid valve. In such an instance, the number of longitudinal struts is a multiple of 3 (e.g., the stent structure 100 comprises 3, 6, 9, 12, 15, 18, or 21 longitudinal struts).

At least some of the longitudinal strut segments 132 of the inner wall 104 may define one or more perforations 144 extending therethrough. The perforations 144 are configured to facilitate attaching (e.g., sewing, stitching, suturing, riveting, clipping, stapling) the leaflets of the valve structure (e.g., leaflets 668 of the valve structure 664 illustrated in FIG. 6) to the longitudinal struts. In an example, the longitudinal strut segments 132 that define the perforations 144 may be positioned closer to the first opening 116 of the inner lumen 102 than the second opening 118 since, generally, it has been found to be more beneficial to position the leaflets closer to the first opening 116 than the second opening 118. In an example, the perforations are formed in every second longitudinal stent when the valve comprises two leaflets, every third longitudinal stent when the valve comprises three leaflets, and so forth to prevent or at least inhibit unequal wear between the leaflets. In an example, each longitudinal strut segment 132 that defines the perforations 144 comprises a plurality of perforations 144. In an example, the portion of the longitudinal stent segment 132 defining the perforations 144 may exhibit a width that is greater than the rest of the longitudinal stent segments 132 thereby allowing the longitudinal stent segment 132 to accommodate larger perforations 144.

A lateral strut may form a partial or complete circumferential or perimeter around a wall of the stent structure 100. To facilitate the expansion and contraction of the overall stent structure 100, one or more of the lateral strut segments 134, or all of the lateral strut segments 134, may comprise a pair of angled legs. Each lateral end of each angled leg is contiguous or integrally formed with a longitudinal strut segment 132 and each angled leg is joined together centrally to form a bend region. While the bend configuration formed by the two angled legs may comprise a simple bend, in other examples, each leg may extend centrally to form a hairpin bend region.

The stent structure 100 may comprise any suitable number of lateral stents. In an embodiment, the number of lateral stents may depend on the desired flexibility of the wall that comprises the lateral stents and the length of the wall. For example, the first region 128 may comprise fewer lateral stents than the adjacent portions of the inner wall 104 because the first region 128 may expand more than the inner wall 104 when expanding from the collapsed configuration. In an embodiment, the number of lateral stents may depend on the stent configuration of the lateral stents. For example, the stent configuration of the lateral stents of the second region 130 allows more flexibility than the stent configuration of the lateral stents of the first region 128. As such, the second region 130 may comprise more lateral stents (e.g., three) than the first region 128 (e.g., one).

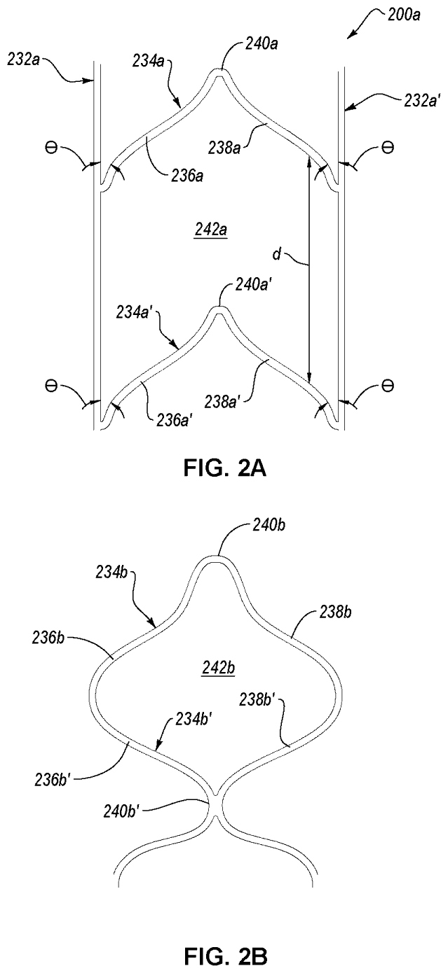

The lateral strut segments may form different stent configurations (e.g., different stent structures). In FIG. 2A, a portion of a stent configuration 200a is illustrated showing an exemplary configuration of a lateral strut segment, according to an embodiment. In the illustrated embodiment, the stent configuration 200a comprises a first longitudinal strut segment 232a and a second longitudinal strut segment 232a′. The stent configuration 200a also comprises a first lateral strut segment 234a and a second lateral strut segments 234a′. The first lateral strut segments 234a comprises a first leg 236a extending from the first longitudinal strut segment 232a and a second leg 238a extending from the second longitudinal strut segment 232a′. The first and second legs 236a, 238a are joined together centrally at a bend region 240a. The second lateral strut segments 234a′ comprises a first leg 236a′ extending from the first longitudinal strut segment 232a and a second leg 238a′ extending from the second longitudinal strut segment 232a′. The first and second legs 236a′. 238a′ are joined together centrally at a bend region 240a′. Longitudinal strut segments 232a, 232a′ and lateral strut segments 234a, 234a′ together form a closed perimeter of a cell 242a.

In some variations, the legs 236a, 238a, 236a′, 238a′ of may comprise a generally linear or straight configuration, with deformations occurring primarily at the intersection between the legs 236a, 238a, 236a′, 238a′ and the longitudinal strut segments 232a, 232a′ and at the bend region 240a, 240a′. In some variations, the legs 236a, 238a, 236a′, 238a′ of may consist of a generally linear or straight configuration. In some variations, legs 236a, 238a, 236a′, 238a′ of may comprise a generally curved configuration.

The first and second lateral strut segments 234a, 234a′ may comprise an acute leg angle θ measured between the linear or substantially linear portions of the legs 236a, 238a, 236a′, 238a′ and the adjacent longitudinal strut segment. For example, the acute leg angle θ may be measured between the linear or substantially linear portion of the first leg 236a of the first lateral strut segment 234a and the first longitudinal strut segment 232a. The acute leg angle θ may vary depending on whether the strut configuration 200a forms part of the inner wall or the outer wall, for instance, because the outer wall exhibits more foreshortening than the inner wall. Generally, the acute leg angle θ is smaller when the stent configuration 200a forms part of the inner wall than when the stent configuration 200a forms part of the outer wall. For example, when the stent configuration 200a forms part of the inner wall, the acute leg angle θ may be 50° or less, 45° or less, 40° or less, 35° or less, 30° or less, 25° or less, 20° or less, 15° or less, or in the ranges of 10° to 20°, 15° to 25°, 20° to 30°, 25° to 35°, 30° to 40°, 35° to 45°, or 40° to 50°. When the stent configuration 200a forms part of the outer wall, the acute leg angle θ may be 30° or greater, 35° or greater, 40° or greater, 45° or greater, 50° or greater, 55° or greater, 60° or greater, 65° or greater, 70° or greater, or in ranges of 30° to 40°, 35° to 45°, 40° to 50°, 45° to 55°, 50° to 60°, 55° to 65°, or 60° to 70.

The first and second lateral strut segments 234a may be separated from each other by a maximum distance d measured parallel to the longitudinal axis of the inner lumen (not shown in FIG. 2A). The maximum distance d may be selected to be 3 mm to 5 mm, 4 mm to 6 mm, 5 mm to 7 mm, 6 mm to 8 mm, 7 mm to 9 mm, 8 mm to 10 mm, 9 mm to 11 mm, or 10 mm to 12 mm. In an embodiment, the maximum distance d may vary depending whether the strut configuration 200a forms part of the inner wall or outer wall since the maximum distance d may affect the flexibility of the walls. Generally, the maximum distance d may be smaller when the strut configuration 200a forms part of the inner wall than when the strut configuration 200a forms part of the outer wall since the inner wall may exhibit less foreshortening than the outer wall.

In the schematic strut configuration 200a depicted in FIG. 2A, the longitudinal strut segments 232a, 232a′ may be parallel or non-parallel, depending on whether the wall comprising the longitudinal strut segments 232a, 232a′ is cylindrical or non-cylindrical (e.g., a frustoconical shape). In variations, where the longitudinal strut segments 232a, 232a′ are non-parallel, the longitudinal struts 232a, 232a′ may have a small radial angle orientation 1° to 5°, 2° to 10°, or 5° to 30° from the longitudinal axis.

In some variations, where greater rigidity is desired, the lateral strut segments 234a, 234a′ may be generally non-uniform along its length. This may be achieved by increasing the relative width of the lateral strut segments 234a, 234a′ near the intersection between the legs 236a, 238a, 236a′, 238a′ and the adjacent longitudinal strut segment and decreasing the relative width the lateral strut segments 234a, 234a′ at or near the bend regions 240a, 240a′.

In some variations, the bend regions 240a, 240a′ may comprise a simple angle or curved configuration. In other variations, the bend regions 240a, 240a′ may comprise arcuate structures having a greater curvature on the same side as an acute angle of the lateral strut segment and the lesser curvature found on an obtuse side of the lateral strut segment.

In some embodiments, the orientations of the lateral strut segments 234a, 234a′ may vary. In an example, as shown in FIG. 2A, the lateral strut segments 234a, 234a′ may be oriented such that the lateral strut segments 234a, 234a′ are generally parallel. In such an example, the bend regions 240a, 240a′ may oriented (e.g., point) in the same direction and the cell 242a may exhibit a chevron-like shape. The bend regions 240a, 240a′ may be oriented in an upstream or downstream direction. Orienting the lateral strut segments 134a, 234a′ to be parallel may prevent the lateral strut segments 234a, 234a′ from contacting each other when the stent configuration 200a is in the collapsed configuration since such contact may limit the extent that the stent configuration 200a may be contracted. In an example, the lateral strut segments 234a, 234a′ may be oriented such that the lateral strut segments 234a, 234a′ are not parallel. In such an example, the bend regions 240a, 240a′ may oriented (e.g., point) in different directions and the cell 242a may exhibit an hourglass or diamond-like shape. In an example, the lateral strut segments 234a, 234a′ may be oriented such that the bend regions 240a, 240a′ point away from the terminal ends of their respective wall that is closest to the lateral strut segments 234a, 234a′. For instance, the bend regions 240a, 240a′ may be oriented to point away from the first opening or the second opening of the inner lumen (e.g., first or second openings 116, 118) when the strut configuration 200a forms part of the inner wall or away from the outer junction or the outer opening when the stent configuration 200a forms part of the outer wall. Orienting the bend regions 240a, 240a′ to point away from the terminal ends of their respective wall may prevent the bend regions 240a, 240a′ from protruding from the rest of the stent structure when in the collapsed configuration. It is noted that, in some embodiments, as shown in FIG. 1D, the lateral stent segment may be sufficiently offset from the terminal end of the wall that the lateral stent segment is unlikely to protrude from the rest of the stent.

FIG. 2B depicts another exemplary embodiment of a stent configuration 200b. Except as otherwise disclosed herein, the stent configuration 200b is the same as or substantially similar to the stent configuration 200a. For example, the stent configuration 200b comprises a first lateral strut segment 234b and a second lateral strut segments 234b′. The first lateral strut segments 234b comprises a first leg 236b and a second leg 238b that are joined together centrally at a bend region 240b. The second lateral strut segments 234b′ comprises a first leg 236b′ and a second leg 238b″ that are joined together centrally at a bend region 240b″. The lateral strut segments 234a, 234b together form a closed perimeter of a stent opening or cell 242b. The stent configuration 200b may not comprise longitudinal strut segments (as shown) or may comprise longitudinal strut segments.

The legs 236b, 238b′, 236b′, 238b′ of lateral strut segments 234b, 234b′ may comprise a curved or curvilinear configuration in its expanded configuration. For example, each leg 236, 238 may exhibit a generally S-like shape. The generally S-like shape of the legs 236b, 238b′, 236b″, 238b may permit a greater amount of expansion of the strut configuration 200b from the collapsed configuration to the expanded configuration, and/or may distribute more stress and strain more along the entire length of the strut configuration 200b.

Additional examples of strut configurations are disclosed in U.S. Pat. No. 11,197,755 issued on Dec. 14, 2021, the disclosure of which is incorporated herein, in its entirety, by this reference.

Referring back to FIGS. 1A to 1E, whether the lateral strut segments 134 exhibits the strut configuration 200a of FIG. 2A, the strut configuration 200b of FIG. 2B, or any other strut configuration may depend on where the lateral strut segments 134 are located on the strut structure 100. For example, the lateral strut segments 134 of the inner wall 104 may generally exhibit the strut configuration 200a of FIG. 2A because of the relative lower amount of radial expansion that is exhibited by the inner wall 104. In other words, the lateral strut segments 134 of the inner wall 104 may exhibit generally linear configurations with deformations occurring primarily at the intersection between the lateral strut segments 134 and the adjacent longitudinal strut segments 132 and at the bend regions thereof. The bend regions of the lateral strut segments 134 may point away from the nearest terminal end of the inner wall 104. As such, the lateral strut segments 134 may form cells exhibiting chevron, hourglass, or other suitable shapes. The lateral strut segments 134 of the first region 128 may also generally exhibit the strut configuration 200a of FIG. 2A because of the relative lower amount of radial expansion that is exhibited by the first region 128 compared to the second region 130. The lateral strut segments 134 of the second region 130 may generally exhibiting the strut configuration 200b of FIG. 2B which allows the lateral strut segments 134 to remain interconnected even though the second region 130 does not comprise longitudinal struts. The lateral strut segments 134 of the second region 130 also allows the second region 130 to exhibit greater flexibility than the inner wall 104 and the first region 128 of the outer wall 106. The greater flexibility of the second region 130 facilitates switching from the stent structure 100 from the collapsed configuration to the expanded configuration thereof. The greater flexibility of the second region 130 also facilitates the second region 130 conforming to the anatomy thereabout.

In an embodiment, the leg lengths of the lateral strut segments 134 of the inner wall 104 are typically shorter than the leg lengths of the lateral strut segments 134 of the outer wall 106 because of the relative lower amount of radial expansion that is exhibited by the inner wall 104 compared to the outer wall 106.

The spacing between adjacent longitudinal or lateral struts may be equal throughout the stent structure 100 or may be different along the folded stent structure. For longitudinal struts, the number of struts may vary depending on the desired flexibility or radial expansion force desired for the stent structure 100, or based on the desired strut segment width to achieve the desired radial expansion force or flexibility. For lateral struts, a relatively larger spacing may be provided in areas were greater radial expansion and/or reduced expansion force is desired, and small spacing in areas of reduced radial expansion and/or greater expansion force is desired.

The stent structure 100 may comprise one or more barbs 146 that deviate radially outward relative to the adjacent struts. The barbs 146 are configured to penetrate into or otherwise press into the adjacent anatomy. Penetrating or otherwise pressing into the anatomy with the barbs 146 may help secure the stent structure 100 to the anatomy. For example, as previously discussed, the enlarge diameter region of the outer wall 106 provides mechanical interference or resistance to displacement. However, the enlarged diameter region of the outer wall 106 may only provide mechanical interference to resistance displacement of the stent structure 100 in a downstream direction. As such, the enlarge diameter region of the outer wall 106 may prevent displacement of the stent structure 100 when the pressure upstream of the stent structure 100 increases (e.g., when the atrial chamber receives blood from the superior vena cava). However, the enlarged 30) diameter region of the outer wall 106 may not prevent or inhibit displacement thereof when the pressure downstream is increased (e.g., when the ventricular chamber pumps blood through the pulmonary valve). Pressing the barbs 146 into the anatomy may provide mechanic interference or resistance to displacement of the stent structure 100 caused by the increased pressure downstream from the stent structure 100.

The barbs 146 may be located anywhere along and/or around the outer wall 106 of the stent structure 100. In an embodiment, at least some of the barbs 146 may be located in the second region 130 of the outer wall 106 since the second region 130 is likely to contact the anatomy due to, at least, the relatively flexibility of the second region 130. In such an embodiment, the barbs 146 may extend outwardly from the portions of the lateral stent segments 134 that intersection with each other since such portions of the lateral stent segments 134 may exhibit greater rigidity and strength than other portions of the lateral stent segments 134. Alternatively or additionally, the barbs 146 may extend from portions of the lateral stent segments 134 that do not intersect with other lateral stent segments 134, such as at the bend regions of the lateral stent segments 134. It is noted that the foreshortening of the second region 130 increases the number of barbs 146 that may be formed thereon. In an embodiment, at least some of the barbs 146 extend outwardly from the first region 128. The barbs 146 may comprise a length 158 in the range of 1 mm to 10 mm, 2 mm to 8 mm, 3 mm to 6 mm, or 3 mm to 5 mm, for example.

In an embodiment, the barbs 146 may be oriented more towards the outer opening 126 than the outer junction 124. Such orientation of the barbs 146 may facilitate pressing the barbs 146 into the anatomy when the pressure downstream from the stent structure 100 is increased. In an embodiment, the barbs 146 may extend outwardly from the outer wall 106 by 2-10 mm, 3-9 mm, or 4-6 mm. In an embodiment, the number of barbs 146 formed in the stent structure 100 may be 1-10, 5-15, 10-20, 15-25, 20-30, 25-35, 30-40, 35-45, 40-50, 45-55, or 50-60. It is noted that, generally, increasing the number of barbs 146 allows the barbs 146 to more securely anchor the stent structure 100 to the anatomy. The barbs may comprise a generally linear or an arcuate shape.

The stent structure 100, when in the expanded configuration, may comprise one or more of the following characteristics:
1) a net longitudinal stent length (i.e. the maximum distance spanned by the stent along the longitudinal axis 120) in the range of 15-55 mm, 20-50 mm, or 30 to 40 mm;
2) a maximum stent diameter or transverse dimension in the range of 40-80 mm, 50-70 mm, or 55-65 mm;
3) a maximum stent diameter or transverse dimension measured at the outer junction 124 in the range of 15-60, 20-50 mm, or 25-40 mm, and is optionally less than the maximum stent diameter or transverse dimension by 10-40 mm, 15-35 mm, or 20-30 mm;
4) an inner lumen length in the range of 10-50 mm, 15-40 mm, or 20-30 mm;
5) an inner lumen diameter or maximum cross-sectional dimension in the range of 10-40 mm, 15-35 mm, or 26-31 mm;
6) a transitional wall radial width measured between the inner junction 122 and the outer junction 124 in the range of 2-10 mm, 3-9 mm, 4-8 mm, or 5-7 mm;
7) an outer wall longitudinal length measured parallel to the longitudinal axis 120 and from the outer junction 124 to the outer opening 126 in the range of 10-40 mm, 20-35 mm, or 25-30 mm;
8) an outer wall longitudinal length to maximum stent diameter ratio (i.e., outer wall longitudinal length/maximum stent diameter) in the range of 0.35 or 1.0, 0.45 to 0.80, or 0.50 to 0.60;
9) a number of longitudinal struts that is divisible by 3, e.g, selected from a group consisting of one or more of 3, 6, 9, 12, 15, 18, or 21 longitudinal struts;
10) a ratio between the radial distance between the barb tip and the longitudinal axis of the stent structure, and the radial distance between an adjacent longitudinal strut or outer wall segment (excluding the barb) and the longitudinal axis of the stent structure, in the range of 1.1 to 1.5, 1.05 to 1.30, 1.05 to 1.20, or 1.05 to 1.15; and/or
11) an offset between the second inner opening 118 and the outer opening 126 that is positive (i.e. protrudes from the outer opening), neutral (i.e. flush with the outer opening), negative (i.e. recessed from the outer opening), and/or in the range of −4 to −12 mm, −5 to −10 mm, −6 to −9 mm, +1 to +8 mm, +2 to +6 mm, +3 to +5 mm, −3 to +3 mm; +0 to +3 mm, −12 to +5 mm, −6 to +6 mm, or −7 mm to +4 mm.

The stent structure 100 need not be limited so as to require a selection of each characteristic recited above, and single characteristics or a subset of characteristics are also contemplated.

In the embodiment depicted in FIGS. 1A to 1E, the stent structure 100 comprises a plurality of longitudinal struts and a plurality of lateral struts, each in turn comprising a contiguous set of contiguous longitudinal or lateral strut segments, respectively. In the particular example of stent structure 100, nine equally spaced apart longitudinal struts are provided, and seven sets of complete lateral struts along folded stent structure 100. Three sets of closely spaced lateral struts are provided along the inner wall 104, with relatively straight or minimally curved legs and with their bend regions oriented away from the closest terminal end of the inner wall 104. The transition wall 108 comprises no lateral struts with a relatively uniform radius of curvature. The outer wall 106 comprises four sets of lateral struts.

The replacement valve may further comprise one or more skirt materials to one or more regions of the stent structure. The skirt materials may comprise solid, tight weave, or loose knit woven sheet of autologous, homologous or heterologous or artificial material that may be the same or different from the leaflet material of the valve. The skirt material may comprise polytetrafluoroethylene (PTFE), polyester or polyethylene terephthalate (PET) material. In variations comprising open pore materials, the average pore size may be in the size range of about 0.035 mm to 0.16 mm, or 0.05 mm to 0.10 mm, or 0.07 mm to 0.09 mm. The open pore materials may provide greater elasticity or flexibility in regions of the stent structure that undergo greater configuration change. Other regions of the stent may be provided with a solid sheet materials, lacking pores, where elasticity or flexibility are not needed. The skirt material may comprise a single layer or a multi-layer structure, and comprise one or more coatings to modulate thrombus formation, tissue ingrowth, and/or lubricity.

As previously discussed, the stent structures disclosed herein may comprise one or more barbs. In an embodiment, the skirt material does not initially define one or more holes corresponding to the one or more barbs of the stent structure. In such an embodiment, the barbs may puncture the skirt material to form tears in the skirt material through which the barbs may extend. The tears in the skirt material formed by the barbs may comprise sharp or other jagged features that form stress concentrators that weaken the skirt material. In an embodiment, the skirt material initially defines one or more holes corresponding to the one or more barbs of the stent structure. The holes allow the barbs to extend through the skirt material without tearing the skirt material. Unlike the tears, the holes may be substantially free of sharp or jagged features that may form stress concentrators thereby significantly decreasing the likelihood that the skirt material fails during use. In an embodiment, the skirt material does not initially define one or more holes corresponding to the one or more barbs of the stent structure. In such an embodiment, as previously discussed, the barbs may form tears in the skirt material. After forming the tears, the portions of the skirt material defining the tears may be heated (e.g., melted) to reduce the number of stress concentrators formed by the tears.

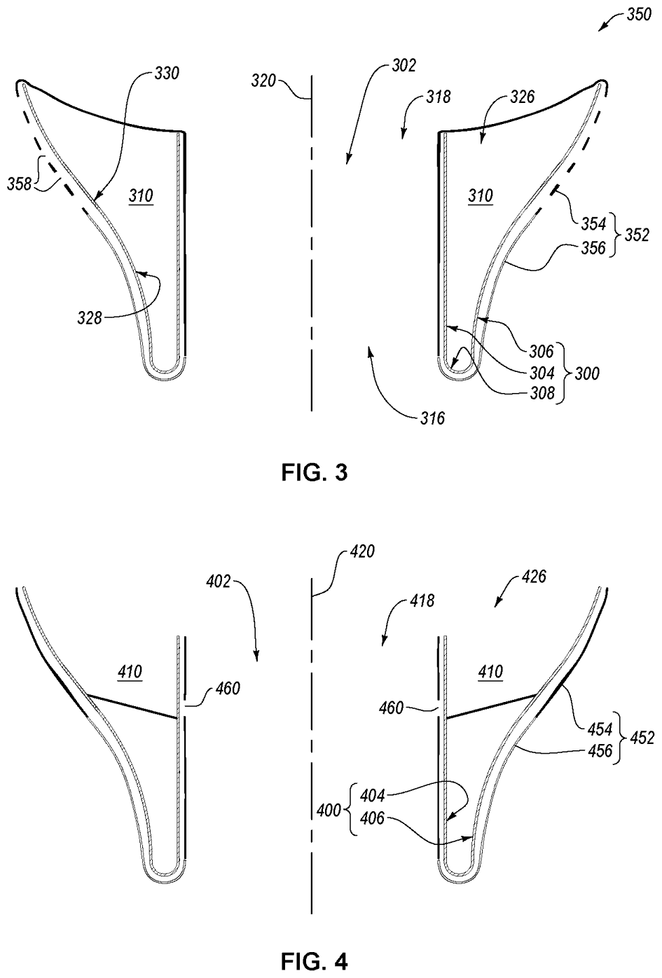

FIG. 3 is a cross-sectional schematic of a replacement valve 350 comprising a stent structure 300 and a skirt material 352, according to an embodiment. Except as otherwise disclosed herein, the replacement valve 350 is the same as or substantially similar to any of the replacement valves disclosed herein. The skirt material 352 comprises two different materials attached to the stent structure 300, a weave material 354 and a knit material 356. The weave material 354 comprises a solid or tight weave material. The weave material 354 is disposed on at least a portion (e.g., all or majority) of the inner wall 304 that defines the inner lumen 302 and at least a portion of the second end 330 of the outer wall 306. The weave material 354 may also extend across the outer opening 326. The weave material 354 may be substantially impermeable or at least more impermeable to blood than the knit material 356. In other words, the weave material 354 forms a barrier to blood flow. The weave material 354 extending across the outer opening 326 may direct the blood from an upstream location through the first opening 316 of the inner lumen 302 and into the inner lumen 302. For example, the weave material 354 extending across the outer opening 326 may be angled relative to the longitudinal axis 320 to form a funnel that directs blood into the inner lumen 302. The weave material 354 disposed in the inner lumen 302 prevents or at least inhibits blood from flowing out of the inner lumen 302 except through the second opening 318 of the inner lumen 302. The weave material 354 disposed across the outer opening 326 and disposed on the second region 330 of the outer wall 306 substantially prevents blood backflowing from a location downstream of the replacement valve 350 to a location upstream of the replacement valve 350 since the second region 330 generally abuts the anatomy of the individual. As such, the weave material 354 may direct the flow of blood through the replacement valve 350 and may prevent backflow of the blood through the replacement valve 350.

The knit material 356 may be disposed on the remainder of the stent structure 300 that is not covered by the weave material 354. For example, the knit material 356 may be disposed on the transition wall 308 and at least a portion of the first region 328 of the outer wall 306. Optionally, the knit material 356 may also be disposed adjacent to portions of the inner wall 304 and the second region 330 that are not covered by the weave material 354, such as a portion of the inner wall 304 downstream from the valve structure (not shown). The knit material 356 exhibits a blood permeability that is greater than the weave material 354. The increased blood permeability of the knit material 356 provide for cellular migration and tissue ingrowth into the replacement valve 350. The increased blood permeability of the knit material 356 may also allow blood to flow into the annular cavity 310 thereby inhibiting or at least decreasing a pressure differential between the annular cavity 310 and a location downstream of the replacement valve 350 that may cause the stent structure 300 to collapse. It is noted that the knit material 356 may exhibit a porosity that is small enough to resist the passage of thrombus that might have formed in the annular cavity 310.

The knit material 356 may exhibit a flexibility that is greater than the weave material 354. As such, inclusion of the knit material 356 in the skirt material 352 better facilitates expansion of the stent structure 300 from the collapsed configuration thereof than if the skirt material 352 only comprised the weave material 354. However, the weave material 354 is disposed on portions of the stent structure 300 (e.g., the second region 330) that expand the most when the stent structure 300 expands from the contracted configuration thereof. In an embodiment, the portions of the weave material 354 disposed on the second region 330 may define one or more slits 358 (schematically shown in FIG. 3 using small gaps). The slits 358 may at least partially extend across the cells formed in the stent structure 300. The slits 358 extending across different cells may be contiguous (i.e., a single slit 358 extends across multiple cells) or discontinuous (i.e., each slit 358 only extends across a single cell). The slits 358 allow the stent structure 300 to freely contract and expand. For example, the cells of the stent structure 300 may elongate vertically when the stent structure 300 is in the collapsed configuration. The slits 358 prevent the relatively rigid weave material 354 from restricting such elongation of the cells since the slits 358 allows the weave material 354 to separate. The slits 358 may be configured to overlap or otherwise be substantially closed when the stent structure 300 is expanded thereby inhibiting blood flowing through the slits 358.

FIG. 4 is a cross-sectional schematic of a replacement valve 450 comprising a stent structure 400 and a skirt material 452, according to an embodiment. Except as otherwise disclosed herein, the replacement valve 450 is the same as or substantially similar to any of the replacement valves disclosed herein. For example, the skirt material 452 may comprise a weave material 454 and a knit material 456.

The weave material 454 may be disposed on at least a portion of the inner wall 404 defining the inner lumen 402 and at least a portion of the second region 430 of the outer wall 406. The weave material 454 does not extend across the outer opening 426. Instead, the weave material 454 extends between a portion of the inner wall 404 and a portion of the second region 430 at an intermediate location that is spaced from the second opening 418 and the outer opening 426. The weave material 454 extending across the intermediate location allows blood to flow from a location upstream of the replacement valve 450 and into a portion of the annular cavity 410 thereby minimizing a pressure differential between the upstream location and the annular cavity 410 that may otherwise cause the weave material 454 to rip. It is noted that the weave material 454 extending across the intermediate location may abut a portion of the second region 430 whose opposing side has the weave material 454 disposed thereon to prevent backflow of the blood. In an embodiment, the portion of the weave material 454 extending across the intermediate location may be angled relative to the longitudinal axis 420 to form a funnel that directs blood into the inner lumen 402. In such an embodiment, the portion of the weave material 454 disposed in the inner lumen 402 that is adjacent to the portion of the weave material 454 extending across the intermediate location may define one or more holes 460 thereby allowing the blood to flow from the annular cavity 410 and into the inner lumen 402. The knit material 456 may be disposed on the portions of the stent structure 400 that are not covered by the weave material 454.

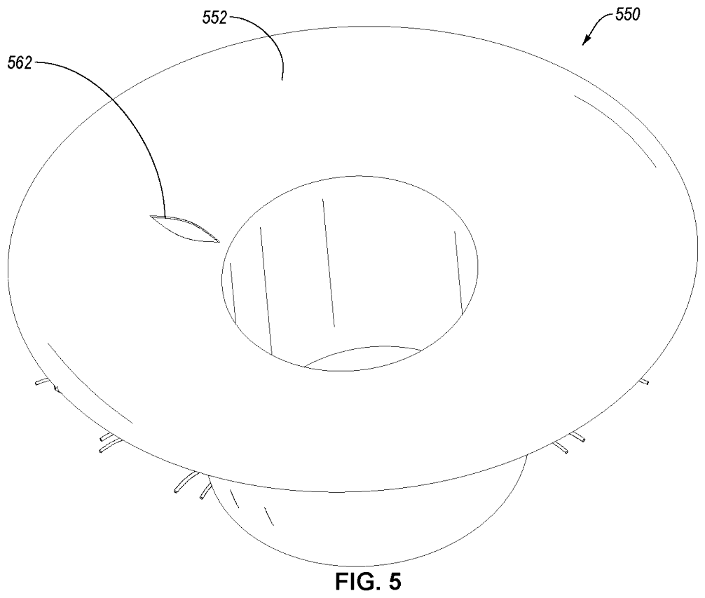

FIG. 5 is an isometric view of a replacement valve 550, according to an embodiment. Except as otherwise disclose herein, the replacement valve 550 is the same as or substantially similar to any of the replacement valves 550 disclosed herein. For example, the replacement valve 550 may comprise a stent structure (not shown, obscured) and a skirt material 552.

The skirt material 552 may define one or more lead openings 562 configured to allow electrical leads (e.g., wires) to extend through the replacement valve 550. For example, depending on the location of the replacement valve 550, the replacement valve 550 may be positioned in a passageway through which electrical leads from a pacemaker or other electrical leads extend in the heart. The lead openings 562 formed in the skirt material 552 may allow these electrical leads to pass through the replacement valve 550 thereby preventing the replacement valve 550 from interfering with the passage of such electrical lead.

The lead openings 562 may comprise any suitable opening. In an embodiment, the lead openings 562 may comprise a hinge door, a trap door, a permeable and/or piercable membrane, or a duck bill port since these structures may prevent or at least inhibit blood from backflowing through the lead openings 562. In an embodiment, the lead openings 562 may be formed in a portion of the skirt material 552 at least one of extending across the outer opening (as shown), disposed on the inner wall of the stent structure, disposed on the transition wall of the stent structure, disposed on the outer wall (e.g., first or second region) of the stent structure, or extending across an intermediate location spaced from the outer opening.

As previously discussed, the replacement valves disclosed herein may comprise a valve structure. FIG. 6 is an isometric view of a replacement valve 650 comprising a valve structure 664, according to an embodiment. Except as otherwise disclosed herein, the replacement valve 650 is the same as or substantially similar to any of the replacement valves disclosed herein. For example, the replacement valve 650 comprises a stent structure (not shown, obscured) and a skirt material 652.

The replacement valve 650 may be configured such that blood flow may be received in through the second opening 618, through the inner lumen 602, and out the first opening (not shown, obscured). As such, the replacement valve 650 may comprise the valve structure 664 attached to the inner lumen 602. The valve structure 664 may be any of a variety of valve structures, comprising a flap valve, ball-in-cage valve, or a leaflet valve. Where a leaflet valve is provided, the valve structure 664 comprises a plurality of leaflets 668. The leaflets 668 may comprise an autologous, homologous or heterologous or artificial material, e.g, a natural material or anatomical structure, such as porcine, bovine or equine pericardial tissue or valve, or biomaterials derived from the patient's own cells, and may be fixated with any of a variety of chemicals, such as glutaraldehyde, to decrease the antigenicity of the valve and/or to alter the physiological and/or mechanical properties of the valve materials. The leaflet valve may be a bi-leaflet or tri-leaflet valve structure. As previously discussed, leaflet valves 668 may be attached or sutured to the longitudinal and/or lateral struts of the inner lumen 602 using the perforations.

### Manufacturing

In some variations, the stent structure may be manufactured using a super-elastic nitinol tube that is laser cut with various slits and slots to achieve the initial tubular stent shape. Next, in a series of cyclic deformation, heating, and cooling steps, the tubular stent is expanded stepwise to at least the initial size of the inner lumen of the stent structure. Then the portion of the stent structure corresponding to the transition wall and outer wall are than further expanded stepwise to the desired diameter, and followed by the a stepwise eversion to form the outer wall using a mandrel is performed to achieve the shape of the outer wall. In another step, one or more bend regions on the lateral struts about the middle region are radially displaced outward to form the barbs.

In an alternate embodiment, after initial cutting the tube, the tube may undergo a series of cyclic deformation, heating, and cooling steps, to expand the tube in a stepwise manner to at least the initial size of the outer lumen of the stent structure, then the portion of the stent structure corresponding to the transition wall and inner wall are then inverted into the outer wall to form the closed end and the inner wall. The outer wall may be further expanded or adjusted stepwise to the desired shape, e.g, by further expanding the open and closed end regions of the outer wall, or by reducing the cross-sectional size or diameter of the middle region. One or more bend regions on the lateral struts about the middle region may also be radially displaced outward to form the retention barbs or structures

### Valve Loading and Delivery

In an embodiment, one or more sutures (e.g., tensioning members) may be attached to a replacement valve (e.g., to the stent structure of the replacement valve) to control the expansion and contraction of different regions on the stent structure until final deployment at the treatment site. In other embodiments, a suture or wrap may be provided over the exterior of one or more regions of the stent structure.

In some examples, the sutures may be tensioned or cinched to collapse the outer and inner walls of the stent structure, for loading onto the delivery catheter. The sutures may be manipulated to collapse inner wall first, before the outer wall, or may collapse both simultaneously. Similarly, one end of the inner wall or outer wall may be collapsed first, or both ends of the inner wall or outer wall may be collapsed simultaneously. This may be done at room temperature, or in a sterile cold or ice water bath at the point of use or at the point of manufacture. After collapse, a sheath may be extended distally over the distal catheter portion where the replacement valve resides. The valve may also be rinsed in sterile saline before loading to remove any remaining preservative on the valve.

In some variations, the transition wall of the stent structure folds down at the inner junction such that in the collapsed configuration, the transition wall is positioned directly over the delivery catheter or tool, like the inner wall, but in other examples, the outer wall is pulled distally during collapse and loading, and unfolds the transition wall at the outer junction, such as the transition wall is located radially outward from the inner wall when contracted into the collapsed configuration.

The retaining sutures of the delivery system may be controlled proximally by the user with pulling rings, sliding levers, and/or rotating knobs, which are further configured to lock into place except during movement via bias springs or mechanical interfit locking configurations as known in the art. The proximal end of the delivery system may also be controlled robotically, using any of a variety of robotic catheter guidance systems known in the art. The sutures may slide along one or more interior lumens of the delivery catheter, in addition to any flush lumen, guidewire lumen, or steering wire lumen(s) provided, comprising rapid exchange guidewire configurations. The sutures may exit at different locations about the distal region of the delivery catheter, and may exit about the distal region of the catheter via multiple openings. The multiple openings may be spaced apart around the circumference of the catheter body and/or spaced apart longitudinally, depending on the region of the stent structure controlled by sutures.

In one exemplary method of delivering the replacement valve, the patient is positioned on the procedure table, and the draped and sterilized in the usual fashion. Anesthesia or sedation is achieved. Percutaneous or cutdown access to the femoral vein is obtained and an introducer guidewire is inserted. A guidewire is manipulated to reach the right atrium. Alternatively, image guidance may be used to detect whether a patent septum ovale or remnant access is available, and the guidewire may be passed through the pre-existing anatomical opening. An electrocautery catheter may also be used to form an opening in the intra-atrial septum. Once in the right atrium, the guidewire is passed through the tricuspid valve.

Referring to FIG. 7A, the delivery system 770 with the delivery catheter 772 and valve 750 is positioned across the tricuspid valve opening 776. For clarity, only the stent structure of the valve 750 is illustrated in FIG. 7A. The delivery system 770 may also be further manipulated to adjust the angle of entry through the tricuspid valve opening 776 to be roughly orthogonal to the native valve opening and/or to be centered with the tricuspid valve opening 776. Once the desired catheter pose is achieved, the delivery catheter 772 is withdrawn proximally, to expose the collapsed valve 750.

In an embodiment, the set of sutures (not shown) are removed thereby allowing the collapsed valve 750 to expand. All of the set of sutures may be remove simultaneously or may be removed or otherwise partially released in a set order. In some variations, when the valve 750 is incorrectly positioned, the sutures may be re-tensioned to re-collapse the valve 750 to facilitate re-positioning and/or re-orienting of the valve 750. After the valve 750 is correctly positioned and expanded, the sutures can be cut or otherwise released or separated from the valve and the sutures may be withdrawn into the catheter and optionally out of the proximal end of the catheter. The delivery catheter and guidewire can then be withdrawn from the patient and hemostasis is achieved at the femoral vein site.

Allowing the valve 750 to expand may comprise allowing the transition wall 708 and the outer wall 706 of the valve 750 to at least partially expand outward. The expansion of the transition wall 708 and the outer wall 706 helps to further center and orient the valve 750, for example, prior to complete release. Expansion of the outer wall 706 also may expose the barbs expending from the outer wall 706 to contact the anatomy of the tricuspid valve 776. Allowing the valve 750 to expand may also comprise allowing the inner wall 704 to expand which also allows the outer wall 706 to achieve its untethered expansion against the tricuspid valve opening 776. FIG. 7B illustrates that valve 750 fully expanded and correctly positioned in the tricuspid valve opening 776.

While the embodiments herein have been particularly shown and described with references to embodiments thereof, it will be understood by those skilled in the art that various changes in form and details may be made therein without departing from the scope of the embodiments. For all of the embodiments described above, the steps of the methods need not be performed sequentially.

1. A replacement heart valve, comprising:
a unibody stent structure comprising:
a collapsed configuration and an expanded configuration;
an outer wall comprising an enlarged diameter region and a reduced diameter region;
an inner wall defining an inner lumen;
a transition wall between the outer wall and the inner wall; and
a valve structure located in the inner lumen of the inner wall;
wherein the unibody stent structure further comprises a plurality of longitudinal struts and a plurality of lateral struts integrally formed together, each longitudinal strut contiguously located along the inner wall, transition wall and a portion of the outer wall;
wherein a ratio of an axial length of a portion of the outer wall without any of the plurality of longitudinal struts and an axial length of the portion of the outer wall with at least some of the plurality of longitudinal struts is in the range of 1:1 to 1:1.5.

2. The replacement heart valve of claim 1, the replacement heart valve is a tricuspid replacement valve.

3. (canceled)

4. The replacement heart valve of claim 1, the outer wall comprises a first region extending from the transition wall and a second region extending from an open end of the outer wall, the first region comprising the plurality of longitudinal struts and the second region free of the plurality of longitudinal struts.

5. The replacement heart valve of claim 4, wherein the first region comprises at least one of the plurality of lateral struts and the second region comprises at least one of the plurality of lateral struts, the at least one of the plurality of lateral struts of the first region exhibiting a strut configuration that is different than the at least one of the plurality of lateral struts of the second region.

6. The replacement heart valve of claim 5, wherein the at least one of the plurality of lateral struts of the first region comprise legs that are generally linear with deformations near the end of each leg, and wherein the at least one of the plurality of lateral struts of the second region comprise legs exhibiting a generally S-like shape.

7. The replacement heart valve of claim 4, wherein at least a portion of the first region of the outer wall is configured to be disposed in a ventricle of a heart and at least a portion of the second region of the outer wall is configured to be disposed in an atrium of the heart.

8. The replacement heart valve of claim 4, wherein the second region of the outer wall is configured to be more flexible than the first region of the outer wall.

9. The replacement heart valve of claim 1, wherein the outer wall comprises a plurality of barbs extending therefrom.

10. The replacement heart valve of claim 9, wherein:
the outer wall comprises a first region extending from the transition wall and a second region extending from an open end of the outer wall, the first region comprising the plurality of longitudinal struts and the second region free of the plurality of longitudinal struts; and
the plurality of barbs extend from the second region of the outer wall.

11. The replacement heart valve of claim 9, wherein the plurality of barbs are oriented more towards an outer opening of the outer wall than towards the transition wall.

12. (canceled)

13. The replacement heart valve of claim 1, further comprising a skirt material disposed on at least a portion of the outer wall, at least a portion of the inner wall, and at least a portion of the transition wall.

14. (canceled)

15. The replacement heart valve of claim 13, wherein the skirt material comprises a weave material and a knit material.

16. The replacement heart valve of claim 15, wherein the weave material is disposed at least a portion of the inner wall and at least a portion of the outer wall extending from an outer opening of the outer wall, a portion of the weave material extending between the inner wall and the outer wall, and wherein the knit material is disposed on at least a portion of the transition wall and a portion of the outer wall extending from the transition wall.

17. The replacement heart valve of claim 16, wherein the portion of the weave material extending between the inner wall and the outer wall:
extends across the outer opening, or
extends across an intermediate location that is spaced from the outer opening.

18. (canceled)

19. The replacement heart valve of claim 13, wherein:
the outer wall comprises a plurality of barbs extending therefrom; and
the skirt material comprises a plurality of openings formed therein, each of the plurality of openings configured to receive one of the plurality of barbs.

20. (canceled)

21. (canceled)

22. The replacement heart valve of claim 1, wherein an inflow angle between an inlet of the outer wall and an inlet of the inner wall is in the range of 5 degrees to 35 degrees, or in the range of 25 degrees to 35 degrees.

23. (canceled)

24. The replacement heart valve of claim 1, wherein a ratio between a diameter of the inner wall and a diameter of the outer wall at an endpoint of at least one of the plurality of longitudinal struts is in the range of 1:1 to 1:2.

25. (canceled)

26. The replacement heart valve of claim 1, wherein the transition wall has an average radius of curvature in the range of about 1 mm to 5 mm.

27. (canceled)

28. The replacement heart valve of claim 1, wherein the ratio of an axial dimension of a combined inner wall and transition wall to the axial dimension of a combined outer wall and transition wall is in the range of about 1:1 to 1:1.5.

29. (canceled)

30. (canceled)

31. (canceled)

32. The replacement heart valve of claim 1, wherein a ratio between a diameter of the outer wall comprising at least one end of the plurality of longitudinal struts and a maximum diameter of the outer wall is in a range of 1:1.2 to 1:1.14.

33. (canceled)
